(19) United States
(12) Patent Application Publication
(10) Pub. No.: US 2012/0009136 A1
(43) Pub. Date: Jan. 12, 2012

(54) ANTIPERSPIRANT PRODUCTS COMPRISING NATURAL PHOSPHOLIPIDS AND METHODS FOR MANUFACTURING THE SAME

(75) Inventors: Travis T. Yarlagadda, Phoenix, AZ (US); Thomas Doering, Scottsdale, AZ (US)

(73) Assignee: The Dial Corporation, Scottsdale, AZ (US)

(21) Appl. No.: 12/833,141

(22) Filed: Jul. 9, 2010

(51) Int. Cl. A61K 8/58; A61Q 15/00

(52) U.S. Cl. 424/66

(57) ABSTRACT

Antiperspirant products comprising natural phospholipids and methods for manufacturing the same are provided. In an embodiment, an antiperspirant product comprises an active antiperspirant compound and a natural phospholipid. In another embodiment, a method of manufacturing an antiperspirant product comprises combining an active antiperspirant compound and a natural phospholipid at a first temperature to form a mixture, pouring the mixture into molds at a second temperature that is lower than the first temperature, and cooling the mixture to a third temperature that is lower than the second temperature.

### FIELD OF THE INVENTION

The present invention generally relates to antiperspirant products and methods for manufacturing antiperspirant products, and more particularly relates to antiperspirant products comprising natural phospholipids and methods for manufacturing the same.

### BACKGROUND OF THE INVENTION

Antiperspirants are popular personal care products used to prevent or eliminate perspiration and body odor caused by perspiration. Antiperspirant sticks are desired by a large majority of the population because of the presence of active antiperspirant compounds that minimize or prevent the secretion of perspiration by blocking or plugging ducts of sweat-secreting glands, such as those located at the underarms. Antiperspirants typically comprise an active antiperspirant compound in a carrier that permits the antiperspirant product to be applied to the skin by swiping or rubbing the stick across the skin, typically of the underarm. Upon application, the carrier evaporates, releasing the active antiperspirant compound from the antiperspirant product to form plugs in the sweat ducts.

Generally, the amount of the active antiperspirant compound in antiperspirant products has its limits. As a preliminary matter, the active antiperspirant compounds in antiperspirant products can be costly. In addition, the Food and Drug Administration has limited the amount of active antiperspirant compound that can be added to an antiperspirant product before the product is considered “clinical” and available only by prescription. Thus, to enhance the effectiveness of active antiperspirant compounds without adding additional active antiperspirant compounds, release enhancers, which can be less costly, have been used. Release enhancers are activated by moisture, such as residual moisture from a shower or bath or from perspiration. When exposed to moisture, the release enhancers draw the active antiperspirant compound into the moisture, which causes the active antiperspirant compound to block or plug the sweat-secreting glands sooner than it ordinarily would, thus increasing the effectiveness of the antiperspirant product. However, known release enhancers do not have a good “skin feel” to users, that is, the release enhancers cause the antiperspirant products to feel abrasive and/or irritating to the skin.

Accordingly, it is desirable to provide antiperspirant products that have enhanced antiperspirant efficacy and improved skin feel. In addition, it is desirable to provide methods for manufacturing such antiperspirant products. Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Antiperspirant products comprising natural phospholipids and methods for manufacturing the same are provided. In accordance with an exemplary embodiment, an antiperspirant product comprises an active antiperspirant compound and a natural phospholipid.

In accordance with another exemplary embodiment, a method of manufacturing an antiperspirant product comprises combining an active antiperspirant compound and a natural phospholipid at a first temperature to form a mixture, pouring the mixture into molds at a second temperature that is lower than the first temperature, and cooling the mixture to a third temperature that is lower than the second temperature.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The various embodiments contemplated herein relate to an antiperspirant product with enhanced antiperspirant efficacy. Various embodiments also exhibit improved “skin feel”, that is, when applied to the skin of a user, the antiperspirant product exhibits reduced caking and crumbly residue, reduced slipperiness, that is, reduced slip between the underarms, and improved glide. The term “glide” typically is used to denote the perceived friction between the antiperspirant product and the skin. The smoother the glide, or the less friction between the product and the skin, the more desirable the product is to users. It unexpectedly has been found that antiperspirant products that exhibit enhanced antiperspirant efficacy and improved skin feel can be achieved when manufactured to contain a natural phospholipid.

In this regard, in one exemplary embodiment, the antiperspirant product comprises at least one natural phospholipid or phospholipid derivative (herein referred to collectively as a “natural phospholipid”). Phospholipids are the phosphorous-containing lipids found in lecithin. Phospholipids useful in the antiperspirant products contemplated herein include phosphoglycerides and sphingolipids. The phospholipid can be unsaturated or hydrogenated, or a lysophospholipid. They can be derived from egg yolks, soybeans, canola, sunflower, corn, rape seed, and the like. Examples of suitable phospholipids for use in the antiperspirant products contemplated herein include phosphatidylcholine, phosphatidylinositol, phosphatidylethanolamine, phosphatidylserine, phosphatidylglycerol, cardiolipin, combinations thereof, and the like. Commercial phospholipids such as Phospholipon® 80 H, which is a hydrogenated phospholipid water/oil emulsion comprising 80 percent phosphatidylcholine, and Phospholipon® 85 G, which is an unsaturated granular product comprising 85% phosphatidylcholine, are available from Lipoid LLC of Newark, N.J. In an exemplary embodiment, the antiperspirant product comprises a natural phospholipid in an amount of no more than about 6 weight percent (wt. %). In a preferred embodiment, the antiperspirant product comprises a natural phospholipid in an amount of no more than about 3 wt. %.

The various embodiments of the antiperspirant products also comprise a water-soluble active antiperspirant compound. Active antiperspirant compounds contain at least one active ingredient, typically metal salts, that are thought to reduce perspiration by diffusing through the sweat ducts of apocrine glands (sweat glands responsible for body odor) and hydrolyzing in the sweat ducts, where they combine with proteins to form an amorphous metal hydroxide agglomerate, plugging the sweat ducts so perspiration cannot diffuse to the skin surface. Some active antiperspirant compounds that may be used in the antiperspirant product include astringent metallic salts, especially inorganic and organic salts of aluminum, zirconium, and zinc, as well as mixtures thereof. Particularly preferred are aluminum-containing and/or zirconium-containing salts or materials, such as aluminum halides, aluminum chlorohydrates, aluminum hydroxyhalides, zirconyl oxyhalides, zirconyl hydroxyhalides, and mixtures thereof. Exemplary aluminum salts include those having the general formula Al₂(OH)ₐCl_{b} x (H₂O), wherein a is from 2 to about 5; a and b total to about 6; x is from 1 to about 6; and wherein a, b, and x may have non-integer values. Exemplary zirconium salts include those having the general formula ZrO(OH)₂₋ₐClₐ x (H₂O), wherein a is from about 1.5 to about 1.87, x is from about 1 to about 7, and wherein a and x may both have non-integer values. Particularly preferred zirconium salts are those complexes that additionally contain aluminum and glycine, commonly known as ZAG complexes. These ZAG complexes contain aluminum chlorohydroxide and zirconyl hydroxy chloride conforming to the above-described formulas. Examples of active antiperspirant compounds suitable for use in the various embodiments contemplated herein include aluminum dichlorohydrate, aluminum-zirconium octachlorohydrate, aluminum sesquichlorohydrate, aluminum chlorohydrex propylene glycol complex, aluminum dichlorohydrex propylene glycol complex, aluminum sesquichlorohydrex propylene glycol complex, aluminum chlorohydrex polyethylene glycol complex, aluminum dichlorohydrex polyethylene glycol complex, aluminum sesquichlorohydrex polyethylene glycol complex, aluminum-zirconium trichlorohydrate, aluminum zirconium tetrachlorohydrate, aluminum zirconium pentachlorohydrate, aluminum zirconium octachlorohydrate, aluminum zirconium trichlorohydrex glycine complex, aluminum zirconium tetrachlorohydrex glycine complex, aluminum zirconium pentachlorohydrex glycine complex, aluminum zirconium octachlorohydrex glycine complex, zirconium chlorohydrate, aluminum chloride, aluminum sulfate buffered, and the like, and mixtures thereof. In a preferred embodiment, the active antiperspirant compound is aluminum zirconium pentachlorohydrex glycine complex or aluminum zirconium trichlorohydrex glycine complex. In a more preferred embodiment, the antiperspirant product comprises an active antiperspirant compound at an active level of about 8 to about 30 wt. % (USP) of the total antiperspirant product. As used herein, weight percent (USP) or wt. % (USP) of an antiperspirant salt is calculated as anhydrous weight percent in accordance with the U.S.P. method, as is well known in the art. This calculation excludes any bound water and glycerine. In a most preferred embodiment, the antiperspirant product comprises about 15-25 wt. % (USP) aluminum zirconium pentachlorohydrex glycine complex or aluminum zirconium trichlorohydrex glycine complex.

Further included in the antiperspirant product is at least one structurant that facilitates the solid consistency of the antiperspirant stick product. Naturally-occurring or synthetic waxy materials or combinations thereof can be used as such structurants. Suitable structurants, including waxes and gellants, are often selected from fatty alcohols often containing from 12 to 30 carbons, such as stearyl alcohol, behenyl alcohol and sterols such as lanosterol. As used herein, the term “fatty” means a long chain aliphatic group, such as at least 8 or 12 linear carbons, which is frequently not branched (linear) and is typically saturated, but which can alternatively be branched and/or unsaturated. It is possible for the fatty acid to contain a hydroxyl group, as in 12-hydroxystearic acid, for example as part of a gellant combination, and to employ amido or ester derivates thereof.

Other structurants can comprise hydrocarbon waxes such as paraffin waxes, microcrystalline waxes, ceresin, squalene, and polyethylene waxes. Other suitable structurants are waxes derived or obtained from plants or animals such as hydrogenated castor oil, hydrogenated soybean oil, carnabau, spermacetti, candelilla, beeswax, modified beeswaxes, and Montan wax and individual waxy components thereof. It is especially suitable herein to employ a mixture of wax structurants. Suitable mixtures of structurants can reduce the visibility of active antiperspirant compounds deposited on the skin and result in either a soft solid or a firm solid. In an exemplary embodiment, the surfactant(s) comprise about 10 to about 35 wt. % of the total antiperspirant product. In a preferred embodiment, the antiperspirant product comprises a mixture of stearyl alcohol and hydrogenated castor oil. In a more preferred embodiment, the antiperspirant product comprises about 12 to about 25 wt. % stearyl alcohol and about 1.5 to about 7 wt. % hydrogenated castor oil.

The antiperspirant products also may comprise a high refractive index (R.I.) hydrophobic compound. As used herein, the term “high refractive index” means a refractive index of no less than about 1.4. The high R.I. hydrophobic compound also facilitates the minimization and/or prevention of a white residue on the skin by masking the active antiperspirant salt that stays upon the skin upon evaporation of a carrier, described in more detail below. Examples of high R.I. hydrophobic compounds for use in the antiperspirant products include PPG-14 butyl ether, C₁₂-C₁₅ alkyl benzoate, such as Finsolv TN® available from Innospec of the United Kingdom, and phenyl dimethicone. In a preferred embodiment, the antiperspirant product comprises PPG-14 butyl ether and, in a more preferred embodiment, the antiperspirant product comprises PPG-14 butyl ether in an amount of about 5 to about 15 wt. % of the total antiperspirant product.

In another exemplary embodiment, the antiperspirant product comprises one or more suspending agents that facilitate suspension of the active antiperspirant compound in the antiperspirant product, thereby minimizing the amount of active antiperspirant compound that settles out of the antiperspirant product during manufacture. Suitable suspending agents include clays and silicas. Examples of suitable silicas include fumed silicas and silica derivatives, such as silica dimethyl silylate. Suitable clays include bentonites, hectorites and colloidal magnesium aluminum silicates. In one exemplary embodiment, the antiperspirant product comprises about 0.2 to about 2.5 wt. % suspending agents. In another exemplary embodiment, the antiperspirant product comprises a mixture of silica and silica dimethyl silylate. In a preferred embodiment, the antiperspirant product comprises from about 0.1-0.5 wt. % silica and from about 0.1 to about 2 wt. % silica dimethyl silylate. In another exemplary embodiment, the antiperspirant product does not use suspending agents, but comprises high melting point waxes to prevent settling of the active antiperspirant compounds. Examples of suitable high melting point waxes include hydrogenated castor oils and polyethylenes having various melting points above 65° C.

In addition to the ingredients identified above, the antiperspirant product may comprise additives, such as those used in conventional antiperspirants. These additives include, but are not limited to, fragrances, including encapsulated fragrances, dyes, pigments, preservatives, antioxidants, moisturizers, and the like. These optional ingredients can be included in the antiperspirant product in an amount of from 0 to about 20 wt. %. In a preferred embodiment, the antiperspirant product comprises myristyl myristate, which provides a conditioning effect to the skin.

The antiperspirant product further comprises at least one hydrophobic carrier. An example of suitable hydrophobic carriers includes liquid siloxanes and particularly volatile polyorganosiloxanes, that is, liquid materials having a measurable vapor pressure at ambient conditions. The polyorganosiloxanes can be linear or cyclic or mixtures thereof. The linear volatile silicones generally have viscosities of less than about 5 centistokes at 25° C., while the cyclic volatile silicones have viscosities under 10 centistokes. Preferred siloxanes include cyclomethicones, which have from about 3 to about 6 silicon atoms, such as cyclotetramethicone, cyclopentamethicone, and cyclohexamethicone, and mixtures thereof. The carrier also may comprise, additionally or alternatively, nonvolatile silicones such as dimethicone and dimethicone copolyols, which have from about 2 to about 9 silicon atoms. Examples of suitable dimethicone and dimethicone copolyols include polyalkyl siloxanes, polyalkylaryl siloxanes, and polyether siloxane copolymers.

The antiperspirant product, according to various embodiments, can be prepared by combining the active antiperspirant compound and the natural phospholipid at 65-75° C. to form a mixture, pouring the mixture into molds at about 53° C., and cooling the mixture to room temperature. In a preferred embodiment, various embodiments can be prepared by combining the suspending agents in the carrier. Any suitable form of mixing can be used to combine the ingredients, such as high shear mixing, stirring, agitation, blending, or any combination thereof. The active antiperspirant compound is added to the suspending agents and carrier to form a premix. Mixing continues until the premix is homogenous and fluid in consistency. In another mixing vessel, the structurants and the high refractive index (R.I.) hydrophobic compound, if used, are added and heat not exceeding 85° C. is applied to melt the ingredients. As the ingredients melt, agitation is slowly commenced. Once the mixture is molten, it is cooled to 65-75° C. and the phospholipid is added. The mixture is cooled to 64-69° C., if necessary, and, with continuous agitation, the premix is incrementally added to the mixture until the mixture is homogenous. Additional carrier is added to the mixture with agitation such that the mixture is maintained at a temperature of 60° C. Additives, such as fragrance, dyes, corn starch, etc. are added with mixing while maintaining the mixture at 60° C. The final mixture is cooled to 53° C., poured into molds, and then allowed to cool to room temperature. As used herein, the term “allowed to cool” means exposing the mixture to room temperature for a time sufficient for the mixture to come to room temperature or exposing the mixture to a refrigerator or cooling room, fan, or other cooling mechanism that lowers the temperature of the mixture to room temperature. In another embodiment, the phospholipid can be added to the premix with high sheer mixing to form the homogenous premix and the premix can be added to the molten mixture as described above. A portion of the phospholipid can be added to the premix in addition to a portion being added directly to the molten mixture, or the entire amount of the phospholipid can be added to the premix as an alternative to the addition to the molten mixture.

The following are exemplary embodiments of an antiperspirant product contemplated herein, with each of the components set forth in weight percent of the antiperspirant product. The examples are provided for illustration purposes only and are not meant to limit the various embodiments of the antiperspirant product in any way.

### Example 1

| Ingredient | Wt. % |
|---|---|
| Cyclopentasiloxane | 37.27 |
| Aluminum zirconium | 21.84 |
| pentachlorohydrex GLY | |
| Stearyl Alcohol | 20.00 |
| PPG-14 butyl ether | 9.80 |
| Hydrogenated castor oil | 2.84 |
| Fragrance | 1.60 |
| Phospholipon ® 80 | 3.00 |
| Myristyl Myristate | 1.92 |
| Silica dimethyl silylate | 1.38 |
| Silica | 0.35 |
| Total | 100.00, |

where Phospholipon® 80 is a hydrogenated phospholipid emulsion comprising 80% phosphatidylcholine, available from Newark, N.J.

### Example 2

| Ingredient | Wt. % |
|---|---|
| Cyclopentasiloxane | 35.88 |
| Aluminum zirconium | 20.00 |
| trichlorohydrex GLY | |
| Stearyl Alcohol | 20.43 |
| PPG-14 butyl ether | 11.00 |
| Hydrogenated castor oil | 2.84 |
| Fragrance and Zea Mays | 0.27 |
| Phospholipon ® 90 | 2.00 |
| Myristyl Myristate | 1.92 |
| Silica | 0.16 |
| Silica dimethyl silylate | 0.65 |
| Fragrance | 2.63 |
| Dye | 0.02 |
| Total | 100.00, |

where Phospholipon® 90 is a hydrogenated phospholipid emulsion comprising 90% phosphatidylcholine, available from Lipoid LLC of Newark, N.J.

Both examples were prepared by adding a portion of the cyclopentasiloxane to a mixing container and initiating agitation. With continuous agitation, the silica and the silica dimethyl silylate were added incrementally to the cyclopentasiloxane until the silicas were wetted. Next, utilizing high shear mixing, a premix was formed by incrementally adding the active antiperspirant compound until the premix had a consistently fluid appearance void of any particulates.

In another mixing container, the hydrogenated castor oil, stearyl alcohol, PPG-14 butyl ether, and myristyl myristate were added and heat was slowly initiated to melt the components while agitation was added as the mix became molten. The temperature of the mixture did not exceed 85° C. Once the components were molten, the phospholipid was added at a temperature between about 65 to about 75° C. With continuous agitation, the premix was incrementally added while the mixture was maintained at a batch temperature of from about 64 to about 69° C. Agitation was continued until the mixture was homogeneous. The remainder of the cyclopentasiloxane was added to the mixture with agitation, while the mixture was maintained at a temperature of about 60° C. The fragrance (and dye and Zea Mays corn starch, if used) were added at about 60° C. with mixing, the mixture was cooled to about 53° C., and the mixture was poured into molds and allowed to cool to room temperature.

Accordingly, various embodiments of antiperspirant products containing natural phospholipids have been provided. The antiperspirant products exhibit enhanced antiperspirant efficacy compared to conventional antiperspirant products without natural phospholipids. Various embodiments also exhibit improved “skin feel”, that is, when applied to the skin of a user, the antiperspirant product exhibits reduced caking and crumbly residue, reduced slipperiness, and improved glide compared to conventional antiperspirants without natural phospholipids.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. An antiperspirant product comprising:
an active antiperspirant compound; and
a natural phospholipid.

2. The antiperspirant product of claim 1, wherein the natural phospholipid is one selected from the group consisting of phosphatidylcholine, phosphatidylinositol, phosphatidylethanolamine, phosphatidylserine, phosphatidylglycerol, cardiolipin, and combinations thereof.

3. The antiperspirant product of claim 1, wherein the natural phospholipid is present in an amount of no more than about 6 wt. % of the antiperspirant product.

4. The antiperspirant product of claim 3, wherein the natural phospholipid is present in an amount of no more than about 3 wt. % of the antiperspirant product.

5. The antiperspirant product of claim 1, wherein the active antiperspirant compound is aluminum zirconium trichlorohydrex glycine complex or aluminum zirconium pentachlorohydrex glycine complex.

6. The antiperspirant product of claim 1, wherein the active antiperspirant compound is present in an amount of from about 8 to about 30 wt. % USP of the antiperspirant product.

7. The antiperspirant product of claim 1, further comprising a carrier.

8. The antiperspirant product of claim 1, further comprising a structurant.

9. The antiperspirant product of claim 8, wherein the structurant comprises stearyl alcohol.

10. The antiperspirant product of claim 1, further comprising a high refractive index hydrophobic compound.

11. The antiperspirant product of claim 10, wherein the high refractive index hydrophobic compound comprises PPG-14 butyl ether.

12. The antiperspirant product of claim 1, further comprising a carrier.

13. The antiperspirant product of claim 12, wherein the carrier is cyclopentasiloxane.

14. A method of manufacturing an antiperspirant product, the method comprising the steps of:
combining an active antiperspirant compound and a natural phospholipid at a first temperature to form a mixture;
pouring the mixture into molds at a second temperature that is lower than the first temperature; and
cooling the mixture to a third temperature that is lower than the second temperature.

15. The method of claim 14, wherein the step of combining comprises combining the active antiperspirant compound and the natural phospholipid selected from the group consisting of phosphatidylcholine, phosphatidylinositol, phosphatidylethanolamine, phosphatidylserine, phosphatidylglycerol, cardiolipin, and combinations thereof.

16. The method of claim 14, wherein the step of combining comprises combining the active antiperspirant compound and the natural phospholipid in an amount of no more than about 6 wt. % of the antiperspirant product.

17. The method of claim 16, wherein the step of combining comprises combining the active antiperspirant compound and the natural phospholipid in an amount of no more than about 3 wt. % of the antiperspirant product.

18. The method of claim 14, wherein the step of combining comprises combining the natural phospholipid and aluminum zirconium trichlorohydrex glycine complex or aluminum zirconium pentachlorohydrex glycine complex.

19. The method of claim 14, wherein the step of combining comprises combining the active antiperspirant compound, the natural phospholipid, and a carrier.

20. The method of claim 14, wherein the step of combining comprises combining the active antiperspirant compound, the natural phospholipid, and a structurant.
